(19) United States
(12) Patent Application Publication
(10) Pub. No.: US 2012/0035278 A1
(43) Pub. Date: Feb. 9, 2012

(54) PREPARING AMINOTRIAZINE ALKOXYLATES

(75) Inventors: Andreas Kunst, Ludwigshafen (DE); Sebastien Garnier, Berlin (DE); Lionel Gehringer, Schaffhouse-pres-Seltz (FR); Achim Löffler, Speyer (DE)

(73) Assignee: BASF SE, Ludwigshafen (DE)

(21) Appl. No.: 13/197,036

(22) Filed: Aug. 3, 2011

Related U.S. Application Data
(60) Provisional application No. 61/370,823, filed on Aug. 5, 2010.

(51) Int. Cl. A61K 47/22; C09K 3/00; C09D 175/04; A01N 25/00; C07D 251/34; C08K 5/3492

(52) U.S. Cl. 514/788; 544/219; 524/101; 521/170; 252/182.12

(57) ABSTRACT

The present invention relates to a process for preparing aminotriazine alkoxylates, which comprises at least one aminotriazine alkoxylate (a) being admixed with at least one aminotriazine (b) and reacted with at least one alkylene oxide (c), wherein the aminotriazine alkoxylate (a) is obtainable by reacting at least one aminotriazine (d) with at least one alkylene oxide (e) and/or at least one alkylene carbonate (g), and optionally at least one aminotriazine alkoxylate (f).

The present invention relates to a process for preparing amino-1,3,5-triazine polyetherols (hereinafter referred to as aminotriazine alkoxylates), more particularly 2,4,6-triamino-1,3,5-triazine polyetherols (also known as melamine alkoxylates), and also to the amino-1,3,5-triazine polyetherols obtainable according to the present invention and their uses.

### PRIOR ART

Preparing alkoxylated aminotriazines is known in principle. U.S. Pat. No. 3,812,122 describes a process wherein aminotriazines are reacted with alkylene oxides in the presence of basic catalysts in N,N-dialkylcarboxamides as solvents at 90-200° C.

U.S. Pat. No. 3,328,321 states that melamine alkoxylates are obtainable for example by reaction of melamine with ethylene carbonate or alkylene oxides in the presence of basic catalysts and solvents such as diethylene glycol dimethyl ether, dimethylformamide and others.

U.S. Pat. No. 3,438,986 describes a process for preparing aminotriazine alkoxylates by reacting aminotriazines with alkylene oxides in the presence of aryldiamines as solvents.

GB 1064148 states that the reaction of aminotriazines with alkylene oxides under basic conditions can also be carried out in the presence of aromatic, saturated or unsaturated polyols. However, the use of melamine requires the additional presence of an inert solvent such as dimethyl sulfoxide.

DE 3412082A1 describes the reaction of aminotriazines with alkylene oxides without the presence of a catalyst and without the use of an inert solvent. However, this process utilizes at least one 2- to 6-hydric aliphatic and/or cycloaliphatic alcohol as co-starter.

The processes described in the documents listed above have serious disadvantages. They require either a) the presence of a polar and hence generally also high-boiling, inert solvent (dimethylformamide or dimethyl sulfoxide) which, after the reaction, has to be expensively and inconveniently removed again from the product, and/or b) the presence of at least one hydroxyl- or amino-modified co-starter, leading to a mixture of an aminotriazine alkoxylate and the co-starter alkoxylate in the product. Thus, the aminotriazine alkoxylate contains the co-starter alkoxylate as an impurity. Since the removal of the high-boiling polar solvents is associated with appreciable technical inconvenience and the presence of a co-starter alkoxylate has an adverse effect on product properties, products of this kind have hitherto failed to become established in the market.

It is an object of the present invention to develop an alternative process for preparing aminotriazine alkoxylates which a) can proceed without the use of inert solvents and b) provides aminotriazine alkoxylates that do not contain any other polyetherols as impurities.

### SUMMARY OF THE INVENTION

We have found that this object is surprisingly achieved by the combinations of features in the claims.

The present invention accordingly provides a process for preparing aminotriazine alkoxylates, which comprises at least one aminotriazine alkoxylate (a) being admixed with at least one aminotriazine (b) and reacted with at least one alkylene oxide (c), wherein the aminotriazine alkoxylate (a) is obtainable by reacting at least one aminotriazine (d) with at least one alkylene oxide (e) and/or at least one alkylene carbonate (g), and optionally at least one aminotriazine alkoxylate (f).

The present invention further provides aminotriazine alkoxylates obtainable by the process of the present invention and also the use of aminotriazine alkoxylates obtainable by the process of the present invention for surface-active materials, more particularly surfactants and emulsifiers, dispersants for aqueous systems, for organic solvents or for polyether polyols, solubilizers for water-insoluble compounds (agro or pharma adjuvants), polyetherol component in polyurethane system formulations, preferably in rigid polyurethane foam or polyurethane coating formulations, more preferably in rigid polyurethane foam systems.

In a preferred embodiment of the present invention process for preparing aminotriazine alkoxylates, the aminotriazine alkoxylate (a) is selected from the group comprising reaction products of at least one compound of the following formula:

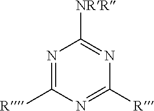

where R′ and R″ are each selected from H, branched or linear C1-C22 compounds, polypropylene glycol and polyethylene glycol, R′″ and R″″ are each selected from H, NR′R″, OH, halide, branched or linear C1-C22 chains, C6R5, where R is selected from H, branched or linear C1-C22 alkyl chains, aryl, or a mixture of compounds having this formula, with at least one alkylene oxide (h).

The aminotriazine alkoxylate (a) of the first reaction batch is obtainable by any one of the methods described above in the cited patent documents.

In addition, the aminotriazine alkoxylate (a) of the first reaction batch is obtainable by the following process steps: 1.) reacting an aminotriazine with aqueous KOH or NaOH solution with subsequent removal of water by vacuum stripping, 2.) reacting the resulting metal salt of the aminotriazine with alkylene oxides, 3.) neutralizing the reaction product (preferably with Brönstedt acids such as phosphoric acid, hydrochloric acid, sulfuric acid, but also CO₂ or Makrosorb® (for example Makrosorb® MPS, a magnesium silicate from INEOS Silicas, specifically developed for purifying polyols)), 4.) removing the neutralization salts from step 3 and unconverted aminotriazine of step 2 from the aminotriazine alkoxylate by filtration. The aminotriazine alkoxylate thus neutralized can be used as starting material (a) in the process described above.

The aminotriazine alkoxylate (a) is obtainable in the presence or absence of a catalyst.

In one embodiment of the process of the present invention, the aminotriazine alkoxylate (a), the starting material of the process of the present invention, is obtained in the presence of a solvent. In this case, it is preferable for residuals of the solvent to be removed, in a conventional manner, before using the aminotriazine alkoxylate (a) in the present invention process for preparing aminotriazine alkoxylates.

In a further embodiment of the process of the present invention, the aminotriazine alkoxylate (a) is obtained in the absence of a solvent.

A person having ordinary skill in the art will know that similar processes are also referred to as “heel processes”, which are characterized in that a reaction batch includes a portion of the reaction product from the previous reaction batch (the “heel”) together with the starting component, in this case the aminotriazine. This process step improves the solubility of the starter, since the reaction product acts as solubilizer, causing the reaction to proceed more completely and not to leave any unconverted aminotriazine behind in the reaction product. Unconverted aminotriazine in the reaction product is a disadvantage because it is present in the reaction product in solid form and phase separates with the liquid reaction product.

In many cases, moreover, no catalyst need be added in the process of the present invention since the amino functionalities of the aminotriazines act autocatalytically for the addition step of the alkylene oxides.

The process of the present invention thus offers an economic advantage, inter alia because no catalyst is required in many cases; in addition, the products can be used in pure form and without additional purification requirements (high in conventional processes because of high-boiling solvents or contamination due to co-starter) directly in the particular application. In contrast to the existing heel processes, moreover, the addition of an alkoxylation catalyst can be dispensed with in this case.

However, since autocatalytic conversion is generally restricted with regard to the molecular weight to be achieved, further reaction of the reaction product from the process described, with alkylene oxide, is also possible in the presence of basic catalysts such as alkali metal hydroxides, alkali metal alkoxides or amines, such as dimethylethanolamine or imidazole.

Aminotriazines useful as aminotriazines (b) and (d) include all aminotriazines comprising at least one and preferably at least two amino groups attached in the molecule. These are for example aminotriazines substituted with aliphatic, cycloaliphatic or aromatic radicals having 1 to 18 carbon atoms, such as: 6-methyl-, 6-ethyl-, 6-n-propyl-, 6-isopropyl-, 6-butyl-, 6-hexyl-, 6-nonyl-, 6-stearyl-, 6-butenyl-, 6-cyclohexyl-, 6-phenyl-, 6-dimethylamino-2,4-diamino-1,3,5-triazine. It is further possible to use aminotriazines having hydroxyl substituents, for example 6-hydroxy-2,4-diamino-1,3,5-triazine (ammeline). Preference is given to using sparingly soluble high-melting aminotriazines such as, for example, 6-methyl-2,4-diamino-1,3,5-triazine, 6-phenyl-2,4-diamino-1,3,5-triazine and, in particular, melamine. The aminotriazines can be used singly or in the form of mixtures.

Aminotriazines (b) and (d) may each also be independently selected from compounds of the following formula:

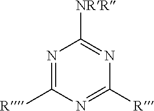

where R′ and R″ are each selected from H, branched or linear C1-C22 compounds, polypropylene glycol and polyethylene glycol, R′″ and R″″ are each selected from H, NR′R″, OH, halide, branched or linear C1-C22 chains, C6R5, where R is selected from H, branched or linear C1-C22 alkyl chains, aryl, or a mixture of compounds having this formula.

Here the aminotriazines (b) and (d) can be the same or different.

Preference is given to 2,4,6-triamino-1,3,5-triazine (also known as melamine) for use as aminotriazine (b) and/or (d).

In a preferred embodiment of the present invention process for preparing aminotriazine alkoxylates, the aminotriazine alkoxylate (f) is selected from the group comprising reaction products of at least one compound of the following formula:

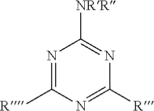

where R′ and R″ are each selected from H, branched or linear C1-C22 compounds, polypropylene glycol and polyethylene glycol, R″' and R″″ are each selected from H, NR′R″, OH, halide, branched or linear C1-C22 chains, C6R5, where R is selected from H, branched or linear C1-C22 alkyl chains, aryl, or a mixture of compounds having this formula, with at least one alkylene oxide (i).

In one embodiment of the process of the present invention, the aminotriazine alkoxylate (f) is present.

In another embodiment of the process of the present invention, the aminotriazine alkoxylate (f) is not present.

The alkylene oxides (c), (e), (h) and (i) are preferably each independently selected from the group comprising propylene oxide, ethylene oxide, 1,2-butylene oxide, 2,3-butylene oxide, isobutylene oxide, 1,2-pentene oxide, styrene oxide, epichlorohydrin, glycidol and mixtures thereof. Similarly, 2,3-pentene oxide, 1,2-hexene oxide, cyclohexene oxide, glycidyl ether and/or butadiene monoxide or mixtures thereof can be used. Particular preference in each case is given to propylene oxide and ethylene oxide.

The alkylene carbonate (g) is preferably selected from the group comprising propylene carbonate, ethylene carbonate, glycerol carbonate and mixtures thereof.

The reaction of the aminotriazines with alkylene oxide in the presence of aminotriazine alkoxylates is preferably carried out at temperatures between 100 and 200° C. and more preferably between 150 and 180° C., generally at pressures of 1-10 bar.

In a preferred embodiment of the process of the present invention, the reaction of aminotriazine alkoxylate (a) with at least one aminotriazine (b) and with at least one alkylene oxide (c) is carried out at temperatures between 100 and 200° C. and at pressures of 1-10 bar in the absence of a catalyst and in the absence of a solvent.

The mass ratio between aminotriazine alkoxylate (a), initially charged to the reactor together with aminotriazine, and the aminotriazine (b) is in the range from 20%:80% to 95%:5% and preferably in the range from 40%:60% to 95%:5%.

The reaction of the aminotriazines with alkylene oxide in the presence of aminotriazine alkoxylates can take place in the presence of basic catalysts. Useful catalysts include for example alkali and alkaline earth metal hydroxides and alkoxides. In addition, amines can be used as catalysts. The use of amines has the technical advantage, particularly with a view to polyurethane applications, that the catalyst does not have to be removed from the end product and accordingly no additional neutralization and filtration step is needed. It is preferable to use tertiary amines. Examples of aminic catalysts are trimethylamine (TMA), tributylamine, triethylamine (TEA), dimethylethanolamine (DMEOA) and dimethylcyclohexylamine (DMCHA), imidazole and substituted imidazole derivatives, preferably dimethylethanolamine.

Particular preference is given to DMEOA (dimethylethanolamine), imidazole.

Carbenes, preferably N-heterocyclic carbenes, can also be used as catalysts.

In a further preferred embodiment, the aminotriazines are reacted with alkylene oxide without the presence of a catalyst. Since the aminotriazines themselves bear catalytically effective functional groups, the reaction can accordingly take place autocatalytically. In this case too there is the technical advantage that there is no need for neutralization and filtration.

After the reaction has ended, the reaction product is generally freed of residual monomer and other volatile components by vacuum stripping. Stripping can be effected for example by means of an inert gas (nitrogen for example) or else with water vapor. When metal hydroxides or metal alkoxides were used as catalysts in the reaction with alkylene oxides, these are for example neutralized by addition of Bronstedt acids and the metal salts separated from the reaction product by filtration.

The alkylene oxides can add blockwise or randomly. Both straight and mixed alkylene oxides can be used.

The reaction of the aminotriazine alkoxylate (a) with at least one aminotriazine (b) and with at least one alkylene oxide (c) to prepare aminotriazine alkoxylates is preferably carried out in the invention without use of an inert solvent.

However, preparing the aminotriazine alkoxylate (a) may, as mentioned, optionally be carried out in the presence of an inert solvent.

According to the present invention, the process for preparing aminotriazine alkoxylates by reacting at least one aminotriazine alkoxylate (a) with at least one aminotriazine (b) and with at least one alkylene oxide (c) preferably does not use any co-starters, for example reactive solvents having hydroxyl or amino groups.

Preparing the aminotriazine alkoxylate (a), however, can optionally be carried out in the presence of a hydroxyl- and/or amino-functional and hence reactive co-starter. The process can be carried out discontinuously as a batch process or else as a semibatch process. The process can also be carried out continuously by continuously feeding the aminotriazine or a mixture of the aminotriazine alkoxylate with the aminotriazine to the reaction tank and continuously removing the product.

The aminotriazine alkoxylates obtainable using the process described generally have a hydroxyl number of 10-600 mg KOH/g, preferably between 50-500 mg KOH/g. The functionalities of the aminotriazines of the present invention are generally between 2-6 and preferably between 3-6. The molecular weights are generally between 300 and 15 000 g/mol and preferably between 400 and 5000 g/mol.

The aminotriazine alkoxylates obtained according to the present invention are useful for a wide variety of purposes, for example for:

1.) formaldehyde-free leather tanning agents and/or leather retanning agents

2.) surface-active materials (surfactants, emulsifiers)

3.) dispersants for aqueous systems, for organic solvents or for polyehter polyols

4.) solubilizers for water-insoluble compounds (agro or pharma adjuvants)

5.) polyetherol component in polyurethane system formulations, preferably in rigid polyurethane foam and polyurethane coating formulations, more preferably in rigid polyurethane foam systems.

In addition, the aminotriazine alkoxylates obtainable by the process of the present invention can be used as an initial charge in a process for preparing further aminotriazine alkoxylates. More particularly, the aminotriazine alkoxylates obtainable by the process of the present invention can also be used as an initial charge in a process for preparing further aminotriazine alkoxylates which is analogous to the process of the present invention. In this case, the aminotriazine alkoxylates obtainable according to the present invention serve in turn as aminotriazine alkoxylate (a) according to claim 1.

The aminotriazine alkoxylates obtainable according to the present invention are as mentioned very useful for forming surface-active agents of the active type. They are accordingly very useful for moistening or softening wool, cotton or cellulose acetate, cellulose nitrate, viscose and similar materials. They are also useful for emulsifying mineral oils, glycerides, fats, oils and the like. The aminotriazine alkoxylates obtainable according to the present invention find application in the formulation of printing inks, dye pastes, dry cleaning baths, leather finishes and flotation agents. By using the aminotriazine alkoxylates obtainable according to the present invention it is possible to ensure bulk, dissolution resistance and enhanced wet strength in the case of rayon and other fibers. The aminotriazine alkoxylates obtainable according to the present invention also reduce the creasing and wrinkling tendency of treated textile materials.

### EXAMPLES

The examples which follow illustrate the invention.

### Example 1

### Preparation of an Aminotriazine Ethoxylate (a1)

46.2 g of melamine were admixed with 1.44 g of aqueous KOH solution (50% strength) in a 300 mL steel autoclave and the reaction mixture was nitrogen inertized three times. Then, the reaction mixture was freed from water by evacuation at 110° C. to 10 mbar. This was followed by the metered addition of 193.8 g of ethylene oxide in the course of 4 hours. After the ethylene oxide kickoff amount had been reached, a postreaction took place until the pressure became constant. After the reaction, the residual monomer was distilled off at 110° C. for 30 min at 10 mbar and the crude product was discharged at room temperature. The crude product additionally contained unconverted melamine in solid form, which was initially separated from the liquid melamine ethoxylate by filtration. The basic filtrate was then admixed with 5% of water and 3% of Makrosorb®, heated at 90° C. for 60 min, freed at 3 mbar and 120° C. from water and other volatile components and finally pressure filtered. The neutralized melamine ethoxylate had a hydroxyl number of 174.0 mg KOH/g and a residual alkalinity of <10 ppm K+(determined by atomic absorption spectrometry, AAS) and was used directly as aminotriazine alkoxylate (a1) for the process of the present invention in accordance with the experimental procedures described hereinbelow.

### Example 2

### Preparation of a Melamine Ethoxylate using the Product of Example 1 as Initial Charge

63 g of the melamine ethoxylate (a1) from Example 1 were initially charged to a 300 mL autoclave together with 7 g of melamine and the mixture was nitrogen inertized three times. Then, the reaction batch was heated to 110° C. and 104 g of ethylene oxide were metered in over 7.5 hours. On completion of the metered addition, a reaction took place for 3 hours. After the reaction had ended, residual monomer was removed in vacuo and the product was discharged at room temperature to obtain 170 g of a homogeneous liquid having a hydroxyl number of 142 mg KOH/g.

### Example 3

### Preparation of a Melamine Ethoxylate using the Product of Example 2 as Initial Charge

63 g of the melamine ethoxylate from Example 2 were initially charged to a 300 mL autoclave together with 7 g of melamine and the mixture was nitrogen inertized three times. Then, the reaction batch was heated to 160° C. and 104 g of ethylene oxide were metered in over 4 hours. On completion of the metered addition, a reaction took place for 3 hours. After the reaction had ended, residual monomer was removed in vacuo and the product was discharged at room temperature to obtain 170 g of a homogeneous liquid having a hydroxyl number of 147 mg KOH/g.

### Example 4

### Preparation of a Melamine Ethoxylate using the Product of Example 3 as Initial Charge

40 g of the melamine ethoxylate from Example 3 were initially charged to a 300 mL autoclave together with 10 g of melamine and the mixture was nitrogen inertized three times. Then, the reaction batch was heated to 160° C. and 171.6 g of ethylene oxide were metered in over 4 hours. On completion of the metered addition, a reaction took place for 3 hours. After the reaction had ended, residual monomer was removed in vacuo and the product was discharged at room temperature to obtain 210 g of a homogeneous liquid having a hydroxyl number of 154.6 mg KOH/g.

### Example 5

### Preparation of a Melamine Ethoxylate using the Product of Example 4 as Initial Charge

28 g of the melamine ethoxylate from Example 4 were initially charged to a 300 mL autoclave together with 12 g of melamine and the mixture was nitrogen inertized three times. Then, the reaction batch was heated to 160° C. and 196 g of ethylene oxide were metered in over 7.5 hours. On completion of the metered addition, a reaction took place for 3 hours. After the reaction had ended, residual monomer was removed in vacuo and the product was discharged at room temperature to obtain 230 g of a homogeneous liquid having a hydroxyl number of 161.3 mg KOH/g.

### Example 6

### Preparation of a Melamine Ethoxylate using the Product of Example 5 as Initial Charge

20.3 g of the melamine ethoxylate from Example 5 were initially charged to a 300 mL autoclave together with 13.5 g of melamine and the mixture was nitrogen inertized three times. Then, the reaction batch was heated to 160° C. and 206.2 g of ethylene oxide were metered in over 10 hours. On completion of the metered addition, a reaction took place for 3 hours. After the reaction had ended, residual monomer was removed in vacuo and the product was discharged at room temperature to obtain 230 g of a homogeneous liquid having a hydroxyl number of 171.2 mg KOH/g.

### Example 7

### Preparation of a Melamine Ethoxylate using the Product of Example 6 as Initial Charge

14.6 g of the melamine ethoxylate from Example 6 were initially charged to a 300 mL autoclave together with 14.5 g of melamine and the mixture was nitrogen inertized three times. Then, the reaction batch was heated to 160° C. and 209 g of ethylene oxide were metered in over 12 hours. On completion of the metered addition, a reaction took place for 3 hours. After the reaction had ended, residual monomer was removed in vacuo and the product was discharged at room temperature to obtain 228 g of a homogeneous liquid having a hydroxyl number of 162.5 mg KOH/g.

### Example 8

### Preparation of a Melamine Ethoxylate using the Product of Example 7 as Initial Charge

14.6 g of the melamine ethoxylate from Example 7 were initially charged to a 300 mL autoclave together with 14.5 g of melamine and the mixture was nitrogen inertized three times. Then, the reaction batch was heated to 160° C. and 209 g of ethylene oxide were metered in over 7.5 hours. On completion of the metered addition, a reaction took place for 3 hours. After the reaction had ended, residual monomer was removed in vacuo and the product was discharged at room temperature to obtain 228 g of a homogeneous liquid having a hydroxyl number of 162.5 mg KOH/g.

### Example 9

### Preparation of an Aminotriazine Propoxylate (a2)

20.0 g of melamine were admixed with 2.4 g of aqueous KOH solution (50% strength) in a 300 mL steel autoclave and the reaction mixture was nitrogen inertized three times. Then, the reaction mixture was freed from water by evacuation at 110° C. to 10 mbar. This was followed by the metered addition of 220.0 g of propylene oxide in the course of 20 hours. After the propylene oxide kickoff amount had been reached, a postreaction took place until the pressure became constant. After the reaction, the residual monomer was distilled off at 110° C. for 30 min at 10 mbar and the crude product was discharged at room temperature. The crude product additionally contained unconverted melamine in solid form, which was initially separated from the liquid melamine propoxylate by filtration. The basic filtrate was then admixed with 5% of water and 3% of Makrosorb®, heated at 90° C. for 60 min, freed at 3 mbar and 120° C. from water and other volatile components and finally pressure filtered. The neutralized melamine propoxylate had a hydroxyl number of 238.4 mg KOH/g and a residual alkalinity of <10 ppm K⁺ (determined by atomic absorption spectrometry, AAS) and was used directly as starting material for the subsequent batches described hereinbelow.

### Example 10

### Preparation of a Melamine Propoxylate using the Product of Example 9 as Initial Charge

106.1 g of the melamine propoxylate (a2) from Example 9 were initially charged to a 300 mL autoclave together with 11.8 g of melamine and the mixture was nitrogen inertized three times. Then, the reaction batch was heated to 160° C. and 122.2 g of propylene oxide were metered in over 9 hours. On completion of the metered addition, a reaction took place for 10 hours. After the reaction had ended, residual monomer was removed in vacuo and the product was discharged at room temperature to obtain 230 g of a homogeneous liquid having a hydroxyl number of 273 mg KOH/g.

### Example 11

### Preparation of a Melamine Propoxylate using the Product of Example 10 as Initial Charge

63.2 g of the melamine propoxylate from Example 10 were initially charged to a 300 mL autoclave together with 15.8 g of melamine and the mixture was nitrogen inertized three times.

Then, the reaction batch was heated to 160° C. and 161 g of propylene oxide were metered in over 12 hours. On completion of the metered addition, a reaction took place for 3 hours. After the reaction had ended, residual monomer was removed in vacuo and the product was discharged at room temperature to obtain 230 g of a homogeneous liquid having a hydroxyl number of 267 mg KOH/g.

### Example 12

### Preparation of a Melamine Propoxylate using the Product of Example 11 as Initial Charge

63.2 g of the melamine propoxylate from Example 10 were initially charged to a 300 mL autoclave together with 15.8 g of melamine and the mixture was nitrogen inertized three times. Then, the reaction batch was heated to 160° C. and 161 g of propylene oxide were metered in over 11 hours. On completion of the metered addition, a reaction took place for 3 hours. After the reaction had ended, residual monomer was removed in vacuo and the product was discharged at room temperature to obtain 230 g of a homogeneous liquid having a hydroxyl number of 270 mg KOH/g.

### Example 13

### Preparation of a Melamine Propoxylate using the Product of Example 12 as Initial Charge

63.2 g of the melamine propoxylate from Example 12 were initially charged to a 300 mL autoclave together with 15.8 g of melamine and the mixture was nitrogen inertized three times. Then, the reaction batch was heated to 160° C. and 140 g of propylene oxide were metered in over 8 hours. On completion of the metered addition, a reaction took place for 3 hours. After the reaction had ended, residual monomer was removed in vacuo and the product was discharged at room temperature to obtain 210 g of a homogeneous liquid having a hydroxyl number of 285 mg KOH/g.

### Example 14

### Preparation of a Melamine Propoxylate using as Initial Charge a Melamine Ethoxylate Prepared by Reaction of Melamine with Ethylene Carbonate

35.2 g of a melamine ethoxylate (a3) having a hydroxyl number of 515.8 mg KOH/g, prepared by reaction of melamine with ethylene carbonate similarly to U.S. Pat. No. 3,328,321, were initially charged to a 300 mL autoclave together with 3.5 g of melamine and the mixture was nitrogen inertized three times. Then, the reaction batch was heated to 100° C. and 100 g of propylene oxide were metered in over 2 hours. On completion of the metered addition, a reaction took place for 3 hours. After the reaction had ended, residual monomer was removed in vacuo and the product was discharged at room temperature to obtain 128 g of a homogeneous liquid having a hydroxyl number of 195 mg KOH/g.

The experimental results accordingly show that the process of the present invention provides homogeneous aminotriazine alkoxylates without use of non-aminotriazine-containing co-starters and without use of an inert solvent. Accordingly, the products are pure aminotriazine alkoxylates, which are obtainable via a simple and economical process.

We claim:

1) A process for preparing aminotriazine alkoxylates, which comprises at least one aminotriazine alkoxylate (a) being admixed with at least one aminotriazine (b) and reacted with at least one alkylene oxide (c), wherein the aminotriazine alkoxylate (a) is obtainable by reacting at least one aminotriazine (d) with at least one alkylene oxide (e) and/or at least one alkylene carbonate (g), and optionally at least one aminotriazine alkoxylate (f).

2) The process for preparing aminotriazine alkoxylates according to claim 1 wherein the aminotriazine alkoxylate (a) is selected from the group comprising reaction products of at least one compound of the following formula: 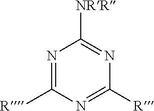
where R′ and R″ are each selected from H, branched or linear C1-C22 compounds, polypropylene glycol and polyethylene glycol, R′″ and R″″ are each selected from H, NR′R″, OH, halide, branched or linear C1-C22 chains, C6R5, where R is selected from H, branched or linear C1-C22 alkyl chains, aryl, or a mixture of compounds having this formula, with at least one alkylene oxide (h).

3) The process for preparing aminotriazine alkoxylates according to claim 1 or 2 wherein the aminotriazine (b) and the aminotriazine (d) are each independently selected from compounds of the following formula: 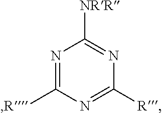
where R′ and R″ are each selected from H, branched or linear C1-C22 compounds, polypropylene glycol and polyethylene glycol, R′″ and R″″ are each selected from H, NR′R″, OH, halide, branched or linear C1-C22 chains, C6R5, where R is selected from H, branched or linear C1-C22 alkyl chains, aryl, or a mixture of compounds having this formula.

4) The process for preparing aminotriazine alkoxylates according to any preceding claim wherein the aminotriazine (b) and/or the aminotriazine (d) is 2,4,6-triamino-1,3,5-triazine.

5) The process for preparing aminotriazine alkoxylates according to any preceding claim wherein the mass ratio between aminotriazine alkoxylate (a) and the aminotriazine (b) is in the range from 20:80 to 95:5 and preferably in the range from 40:60 to 95:5.

6) The process for preparing aminotriazine alkoxylates according to any preceding claim wherein the aminotriazine alkoxylate (f) is selected from the group comprising reaction products of at least one compound of the following formula: 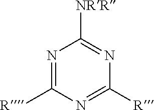
where R′ and R″ are each selected from H, branched or linear C1-C22 compounds, polypropylene glycol and polyethylene glycol, R′″ and R″″ are each selected from H, NR′R″, OH, halide, branched or linear C1-C22 chains, C6R5, where R is selected from H, branched or linear C1-C22 alkyl chains, aryl, or a mixture of compounds having this formula, with at least one alkylene oxide (i).

7) The process for preparing aminotriazine alkoxylates according to any preceding claim wherein the aminotriazine alkoxylate (f) is present.

8) The process for preparing aminotriazine alkoxylates according to any one of claims 1 to 6 wherein the aminotriazine alkoxylate (f) is not present.

9) The process for preparing aminotriazine alkoxylates according to any preceding claim wherein the alkylene oxides (c), (e), (h) and (i) are each independently selected from the group comprising propylene oxide, ethylene oxide, 1,2-butylene oxide, 2,3-butylene oxide, isobutylene oxide, 1,2-pentene oxide, styrene oxide, epichlorohydrin, glycidol and mixtures thereof, preferably propylene oxide or ethylene oxide.

10) The process for preparing aminotriazine alkoxylates according to any preceding claim wherein the alkylene carbonate (g) is selected from the group comprising propylene carbonate, ethylene carbonate, glycerol carbonate and mixtures thereof.

11) The process for preparing aminotriazine alkoxylates according to any preceding claim wherein the aminotriazine alkoxylate (a) is obtained in the presence of a solvent.

12) The process for preparing aminotriazine alkoxylates according to any of claims 1 to 10 wherein the aminotriazine alkoxylate (a) is obtained in the absence of a solvent.

13) The process for preparing aminotriazine alkoxylates according to any preceding claim wherein the reaction of aminotriazine alkoxylate (a) with at least one aminotriazine (b) and with at least one alkylene oxide (c) is carried out at temperatures between 100 and 200° C. and at pressures of 1-10 bar in the absence of a catalyst and in the absence of a solvent.

14) An aminotriazine alkoxylate obtainable by the process of any of claims 1 to 13.

15) The use of aminotriazine alkoxylates obtainable by the process of any of claims 1 to 13 for surface-active materials, more particularly surfactants and emulsifiers, dispersants for aqueous systems, for organic solvents or for polyether polyols, solubilizers for water-insoluble compounds (agro or pharma adjuvants), polyetherol component in polyurethane system formulations, preferably in rigid polyurethane foam or polyurethane coating formulations, more preferably in rigid polyurethane foam systems.
